(19) United States
(12) Patent Application Publication
(10) Pub. No.: US 2007/0129419 A1
(43) Pub. Date: Jun. 7, 2007

(54) Serotonin transporter (SERT) inhibitors for the treatment of depression and anxiety

(76) Inventors: Christophe Grundschober, Rodersdorf (CH); Torsten Hoffmann, Weil am Rhein (DE); Andreas Koblet, Bottmingen (CH); Patrick Schnider, Bottmingen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/603,515

(22) Filed: Nov. 22, 2006

(30) Foreign Application Priority Data
Dec. 1, 2005 (EP) 05111565.7

(51) Int. Cl. A61K 31/40; C07D 207/04

(52) U.S. Cl. 514/408; 548/571

(57) ABSTRACT

The present invention relates to cis-derivatives of formula 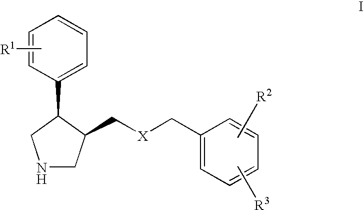 wherein
R¹, R², and R³ are as defined herein and to pharmaceutically acceptable acid addition salts thereof. The compounds of formula I are good inhibitors of the serotonin transporter (SERT inhibitors). By virtue of their efficacy as SERT inhibitors, the compounds of the present invention are particularly useful for the treatment of CNS disorders and psychotic disorders, in particular in the treatment or prevention of depressive states and/or in the treatment of anxiety.

### PRIORITY TO RELATED APPLICATIONS

This application claims the benefit of European Application No. 05111565.7, filed Dec. 1, 2005, which is hereby incorporated by reference in its entirety.

### BACKGROUND OF THE INVENTION

SERT Inhibitors including the selective serotonin transporter inhibitors, also called selective serotonin reuptake inhibitors (SSRI's), have become the most frequently prescribed antidepressant drugs. They are believed to exert their effect by increasing extracellular 5-HT levels in the serotoninergic terminal fields such as the hippocampus and prefrontal cortex. However, approximately 30% of patients appear to be resistant to SSRI treatment. In addition, those patients who do benefit from SSRI treatment often exhibit various side-effects which include sexual dysfunction, gastrointestinal distress, insomnia and in some cases anxiogenesis due to their indirect activation (through elevation of 5-HT levels) of all 5-HT receptors. Furthermore, a common problem in current antidepressant therapies is their slow onset of action, since a delay of about 4 weeks is normally observed between the beginning of the treatment and alleviation of the symptoms. The delay appears to parallel the progressive desensitization of somatodendritic 5HT_{1A} receptors, increasing serotoninergic function, thus allowing alleviation of depressive symptoms.

Therefore, there is a need in the art to develop compounds which have SERT inhibitory activity with an additional beneficial effect on the onset of action and which allow major improvements for SSRI-resistant patients, e.g. with a reduced anxiogenic or even anxiolytic profile.

Recent reports have indicated that the combination of a SSRI and a NK-1 antagonist produces beneficial responses in animal models of anxiety and depression such as guinea-pig pup maternal separation vocalization, with relatively reduced doses (WO98/47514;. Bioorg. Med. Chem. Lett. 12(2), 261-264 (2002) and Bioorg. Med. Chem. Lett. 12(21), 3195-3198 (2002).

This suggests that by adopting a dual approach which is mechanistically dissimilar, a synergism between the two modes of action may occur, enabling enhanced responses. This may not only be beneficial in patients resistant to treatment with SSRI alone but also in improving the rapidity of onset of therapeutic action. A drug with a dual mode of action potentially allows for a reduction in dosing and therefore a decreased risk of side effects as compared to a combination of two drugs.

In the patent literature the combined NK-1/SSRI approach also has been proposed as a potential treatment for obesity (WO98/47514).

WO2005/032464 describes trans-phenyl pyrrolidine ethers which are tachykinin receptor antagonists. All of the examples described in the '464 application have a substitution on the N-atom of the pyrrolidine ring, which seems to be necessary for selective tachykinin receptor antagonistic activity.

### SUMMARY OF THE INVENTION

The present invention provides compounds that have serotonin transporter (SERT) inhibitory activity with an additional beneficial effect on the onset of action and with allowing major improvements for SSRI-resistant patients, e.g., those patients with a reduced anxiolgenic or even anxiolytic profile.

In particular, the invention provides a small group of cis-derivatives of pyrrolidines of formula I 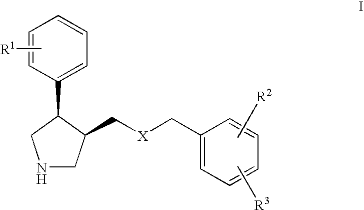
wherein
R¹ is hydrogen, halogen or lower alkyl;
R² and R³ are each independently hydrogen, halogen, CN, lower alkyl, lower alkyl substituted by halogen, lower alkoxy or lower alkoxy substituted by halogen;
X is —O— or —N(R)—; and
R is hydrogen or lower alkyl;
   and pharmaceutically acceptable acid addition salts thereof.

These compounds, which are not substituted on the N-atom of the pyrrolidine ring, are good inhibitors of the serotonin transported. By virtue of their efficacy as SERT inhibitors, the compounds of the present invention are particularly useful for the treatment of CNS disorders and psychiatric disorders, in particular, the treatment of depressive states and/or in the treatment of anxiety.

Concommitantly, these compounds also are NK-1 receptor antagonists. NK-1 antagonists are believed to indirectly modulate 5-HT function via noradrenergic pathways and have been shown to attenuate presynaptic 5HT_{1A} receptor function. (Bioorg. Med. Chem. Lett. 12 (2002) 261-264). A drug with a dual mode of action, such as compounds of formula I, combines the advantage of both receptor sites, allowing for dose reduction, leading to a decreased risk of side effects compared to delivery of a combination of two separate drugs. Thus, the combination of serotonin uptake inhibition with NK-1 antagonism can lead to compounds with an improved onset of action and better efficacy during treatment of depressive/anxiolytic states.

The present invention also provides compositions containing compounds of formula I and a pharmaceutically acceptable carrier. The invention further provides methods for the treatment of diseases related to activation of serotonin transporter (SERT), such as for the treatment of depression and anxiety. The invention also provides processes for the manufacture of cis-pyrrolidine derivatives of formula I.

The compounds of formula I are good inhibitors of the serotonin transporter (SERT inhibitors). By virtue of their efficacy as SERT inhibitors, the compounds in the present invention are particularly useful for the treatment of CNS disorders and psychotic disorders, in particular in the treatment or prevention of depressive states and/or in the treatment of anxiety.

### DETAILED DESCRIPTION OF THE INVENTION

As used herein, the term “lower alkyl” denotes a saturated straight- or branched-chain hydrocarbon group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

The term “halogen” denotes chlorine, iodine, fluorine and bromine.

The term “lower alkyl substituted by halogen” denotes a saturated straight- or branched-chain hydrocarbon group containing from 1 to 7 carbon atoms as described above, wherein at least one hydrogen atom is replaced by a halogen atom, for example CF₃, CHF₂ or CH₂F.

The term “lower alkoxy” denotes a residue O—R, wherein R is a saturated straight- or branched-chain group containing from 1 to 7 carbon atoms as described for “lower alkyl”.

The term “lower alkoxy substituted by halogen” denotes a lower alkoxy group as defined above, wherein at least one hydrogen atom is replaced by a halogen atom, for example OCF₃, OCHF₂ or OCH₂F.

The term “pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

The term “therapeutically effective” denotes an amount that is effective to prevent, alleviate, or ameliorate symptoms of disease or prolong the survival of the subject being treated.

The present invention provides a small group of cis-derivatives of pyrrolidines of formula I 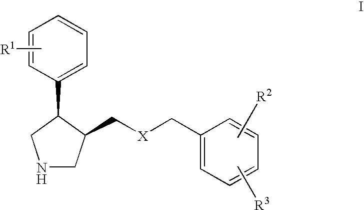
wherein
R¹ is hydrogen, halogen or lower alkyl;
R² and R³ are each independently hydrogen, halogen, CN, lower alkyl, lower alkyl substituted by halogen, lower alkoxy or lower alkoxy substituted by halogen;
X is —O— or —N(R)—; and
R is hydrogen or lower alkyl;
   and pharmaceutically acceptable acid addition salts thereof.

In one embodiment, the invention provides compounds of formula I having formulae IA and IB

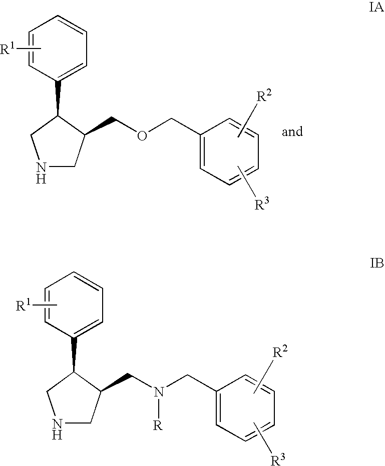

Preferred compounds of formula I are those of formula I-A, for example the following compounds:
(+)-3-(3,5-bis-trifluoromethyl-benzyloxymethyl)-4-(4-fluoro-phenyl)-pyrrolidine,
(+)-3-(3,5-bis-trifluoromethyl-benzyloxymethyl)-4-(3-fluoro-phenyl)-pyrrolidine,
(+)-3-(3,5-bis-trifluoromethyl-benzyloxymethyl)-4-(2-fluoro-phenyl)-pyrrolidine,
(+)-3-(3,5-bis-trifluoromethyl-benzyloxymethyl)-4-(4-chloro-phenyl)-pyrrolidine,
(+)-3-(3,5-bis-trifluoromethyl-benzyloxymethyl)-4-(3-chloro-phenyl)-pyrrolidine,
(+)-3-(3,5-bis-trifluoromethyl-benzyloxymethyl)-4-(2-chloro-phenyl)-pyrrolidine,
(+)-3-(3,5-bis-trifluoromethyl-benzyloxymethyl)-4-o-tolyl-pyrrolidine,
(+)-3-(3,5-bis-trifluoromethyl-benzyloxymethyl)-4-phenyl-pyrrolidine,
(+)-3-(3,5-dibromo-benzyloxymethyl)-4-phenyl-pyrrolidine,
(+)-3-(3,5-dimethyl-benzyloxymethyl)-4-phenyl-pyrrolidine,
(+)-3-(3-fluoro-5-trifluoromethyl-benzyloxymethyl)-4-phenyl-pyrrolidine,
(+)-3-phenyl-4-(3-trifluoromethyl-benzyloxymethyl)-pyrrolidine,
(+)-3-phenyl-4-(3-trifluoromethoxy-benzyloxymethyl)-pyrrolidine,
(+)-3-(3,5-dimethoxy-benzyloxymethyl)-4-phenyl-pyrrolidine, and
(+)-3-phenyl-4-(2-trifluoromethyl-benzyloxymethyl)-pyrrolidine.

Preferred compounds of formula I are further those of formula I-B, for example the following compounds
(+)-(3,5-bis-trifluoromethyl-benzyl)-((3R,4R)-4-phenyl-pyrrolidin-3-ylmethyl)-amine dihydrochloride and
(3,5-bis-trifluoromethyl-benzyl)-methyl-((3R,4R)-4-phenyl-pyrrolidin-3-ylmethyl)-amine dihydrochloride.

In another embodiment, the present invention preferred compounds are those in which X is N(R) and R is hydrogen. Other preferred compounds are those where X is N(R) and R is lower alkyl.

In another embodiment, preferred compounds are those in which R¹ is hydrogen. Other preferred compounds are those in which R¹ is halogen. Still other preferred compounds are those wherein R¹ is lower alkyl, for example those where R¹ is methyl.

In still another embodiment, preferred compounds are those in which at least one of R² and R³ is hydrogen. Other preferred compounds are those in which at least one of R² and R³ is lower alkyl, in particular methyl. Still other preferred compounds are those in which at least one of R² and R³ is halogen.

Preferred compounds of the invention include those in which at least one of R² and R³ is lower alkyl substituted by halogen, for example CF₃. Other preferred compounds include those in which at least one of R² and R³ is lower alkoxy, for example OCH₃. Further preferred compounds are those in which at least one of R² and R³ is lower alkoxy substituted by halogen, for example OCF₃.

The novel cis-derivatives of formula I and their pharmaceutically acceptable salts can be prepared by methods known in the art, for example by processes described below, which processes comprise
a) reacting a compound of formula 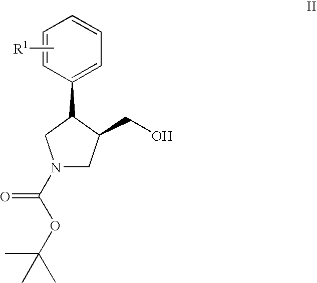
with sodium hydride and a compound of formula 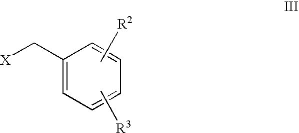
followed by treatment with an acid, such as HCl or trifluoroacetic acid, to give a compound of formula 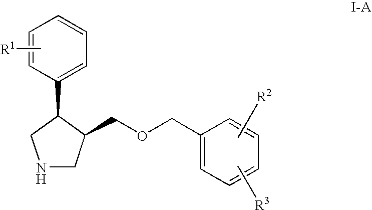
wherein X is Cl, Br or I and R¹ to R³ are as described above, or
b) reacting a compound of formula 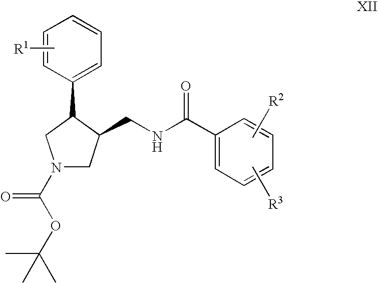
with a THF solution of borane dimethylsulfide complex in toluene and then with an acid, such as HCl or trifluoroacetic acid, to obtain a compound of formula 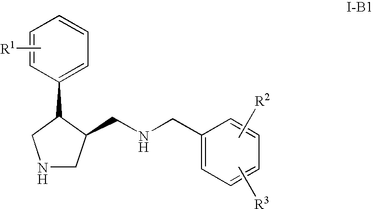
wherein the substituents are as described above, or

c) alkylating a compound of formula 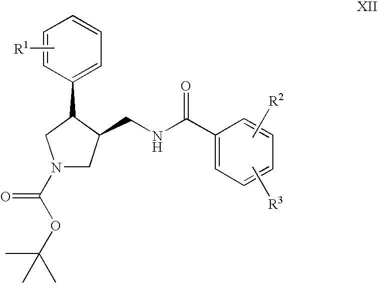
with an alkylating agent, such as methyliodide, to obtain a compound of formula 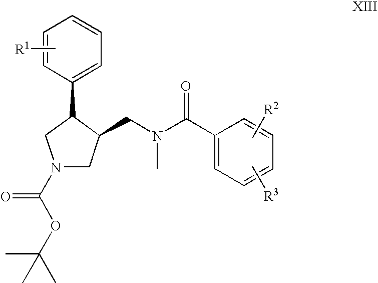
which is consecutively reacted with a THF solution of borane dimethylsulfide complex in toluene and then with an acid, such as HCl or trifluoroacetic acid, to obtain a compound of formula 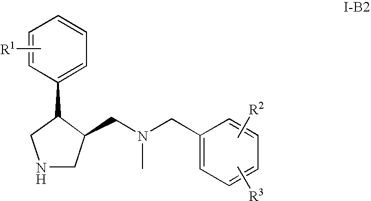
wherein the substituents are as described above, or if desired, converting the compounds obtained into pharmaceutically acceptable acid addition salts.

Schemes I and 2 show the preparation of compounds of formula I in more detail. The starting material used in schemes 1 and 2 are known compounds or may be prepared by methods known in the art.

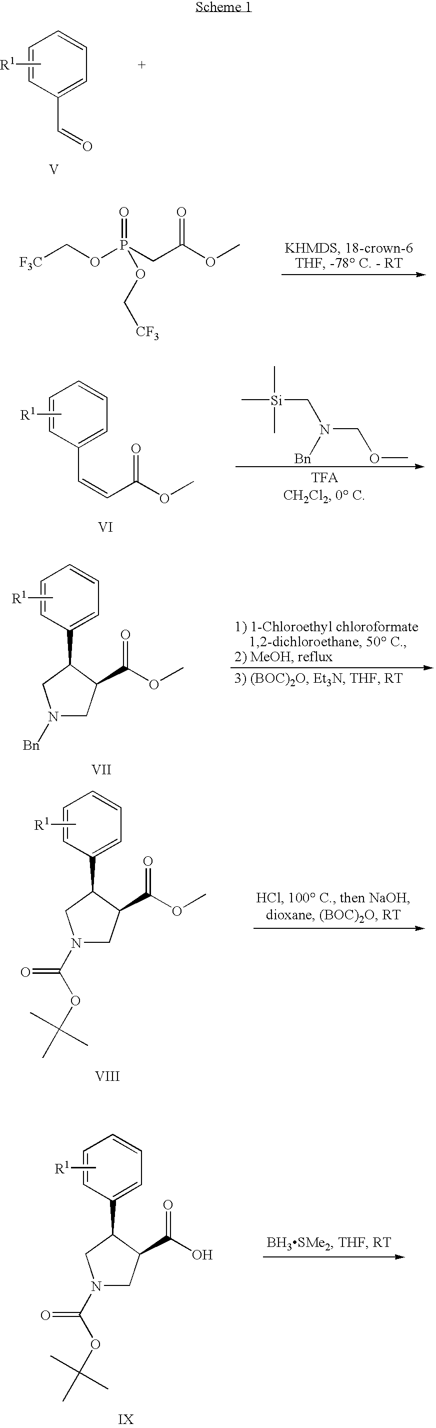

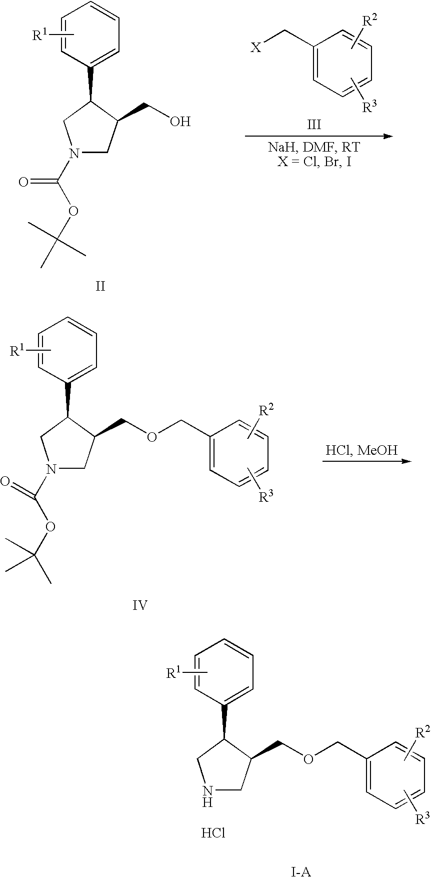

In accordance with scheme 1, compounds of formula I-A may be prepared as follows: To a solution of bis(2,2,2-trifluoroethyl) (methoxycarbonylmethyl)-phosphonate and 18-crown-6 THF is added potassium hexamethyldisilazide solution at −78°. After 5 min a compound of formula V is added. The reaction mixture is stirred at −78° C. for about 20 min and consequently allowed to warm to room temperature over a period of about 1 h, followed by quenching with a saturated aqueous solution of ammonium chloride and by extraction in conventional manner. The obtained compound of formula VI is dissolved in dichloromethane and consecutively treated with N-(methoxymethyl)-N-(trimethylsilylmethyl)benzylamine (96%, Aldrich) and a solution of dichloromethane and trifluoroacetic acid is added at 0° C. Another portion of N-(methoxymethyl)-N-(trimethylsilylmethyl)benzylamine (96%, Aldrich), is added after one hour if necessary to drive conversion to completion. The reaction mixture is diluted with dichloromethane and washed with saturated sodium carbonate solution. After concentration and purification, a compound of formula VII is obtained. A solution of a compound of formula VII and 1-chloroethyl chloroformate in 1,2-dichloroethane is heated at about 50° C. over night. After cooling to room temperature the solvent is evaporated. The residue is redissolved in MeOH, and the resulting solution is heated at reflux for 30 min. The reaction mixture is concentrated in vacuo. The residual hydrochloride salt is redissolved in THF. The mixture is treated with triethylamine and a solution of di-tert.-butyl dicarbonate in THF. After stirring for about 90 min the reaction mixture is worked up in conventional manner to obtain a compound of formula VIII. Then a mixture of a compound of formula VIII and hydrochloric acid solution is heated at reflux for about 4 h. The reaction mixture is cooled to 0° C. and basified by the addition of sodium hydroxide. Dilution with dioxane is followed by addition of a solution of di-tert.-butyl dicarbonate in 1,4-dioxane. The reaction mixture is concentrated and purified in conventional manner to obtain a compound of formula IX.

To a solution of such acid (IX) in THF is added a borane dimethylsulfide complex solution in THF at 0° C. The mixture is stirred for about 15 min at 0° C. and then for 5 h at room temperature. After cooling to 0° C. the reaction is quenched by the addition of methanol. Stirring is continued until no evolution of gas is observed any more. The reaction mixture is concentrated in vacuo and purified to obtain a compound of formula II.

Furthermore, to a solution of a compound of formula II in DMF is added sodium hydride at 0° C. The reaction mixture is allowed to warm to room temperature. After about 1 h a compound of formula III is added. Stirring at room temperature for 2.5 h is followed by quenching with water, extraction and purification. Then a solution of a compound of formula IV in hydrochloric acid and methanol is stirred for 30′ at about 50° C. The reaction mixture is concentrated to dryness to give a compound of formula I-A.

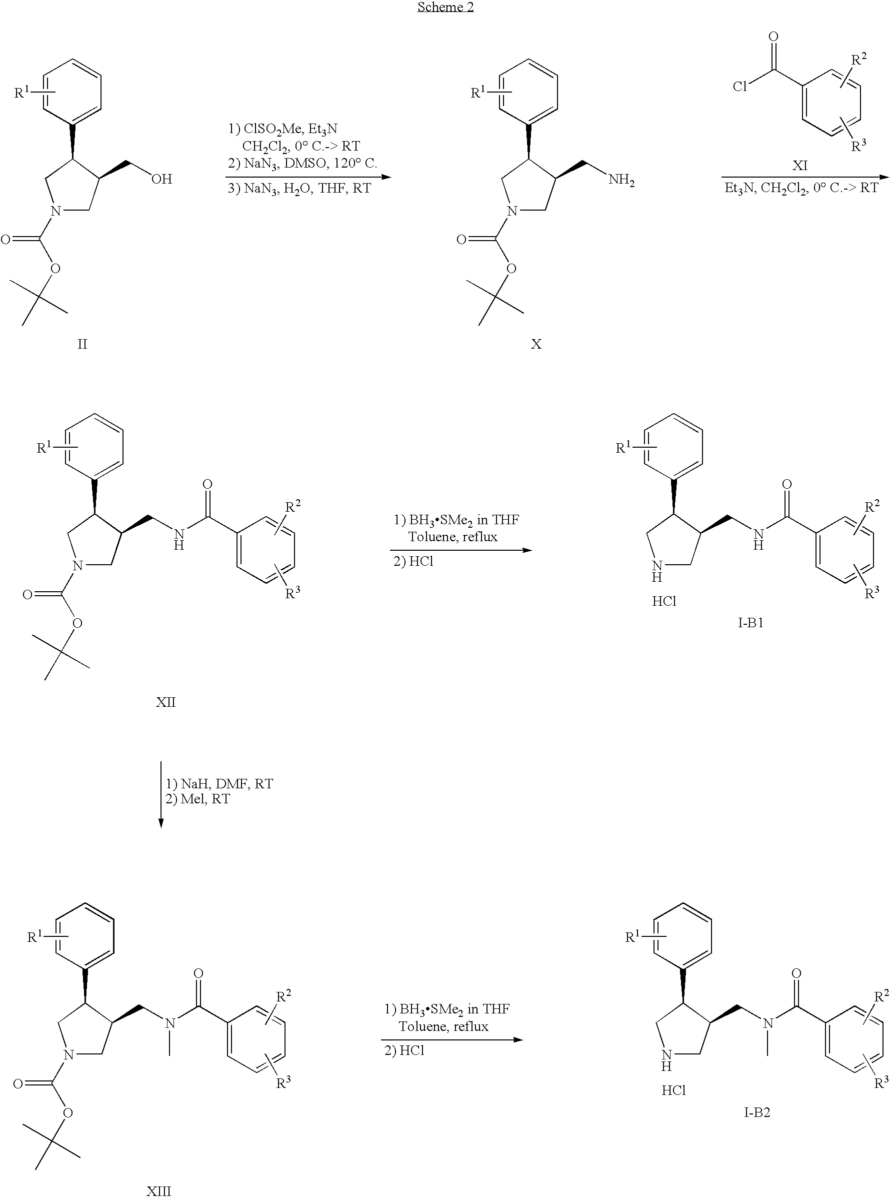

Compounds of formulas I-B1 and I-B2 can be prepared as follows:

To a solution of a compound of formula II and triethylamine in dichloromethane is added dropwise at 0° C. methanesulfonyl chloride. After completed addition the reaction mixture is allowed to warm to room temperature during 30 minutes. Quenching with water is followed by extraction. The obtained crude product is used in the next step without purification. A mixture of this product and sodium azide in dimethylsulfoxide is heated at about 120° C. for 5 h. The mixture is cooled to room temperature, diluted with a aqueous solution of sodium carbonate and extracted with methyl tert.-butyl ether. The combined organic layers are washed and dried to give the corresponding crude 3-azidomethyl-4-phenyl-pyrrolidine-1-carboxylic acid tert-butyl ester. This compound is dissolved in THF and treated with triphenylphosphine and water. After stirring at room temperature for about 20 h the reaction mixture is diluted with methyl tert.-butyl ether, washed and purified to give a compound of formula X.

Then to a solution of a compound of formula X and triethylamine in dichloromethane is added a compound of formula XI at 0° C. After completed addition, the reaction mixture is allowed to warm to room temperature and stirred for one hour. Quenching with water is followed by basification with sodium hydroxide solution, extraction and purification. It is obtained a compound of formula XII.

Then a mixture of a compound of formula XII and of a THF solution of borane dimethylsulfide complex in toluene is heated at reflux for about 20 h. After addition of another portion of a THF solution of borane dimethylsulfide complex, the mixture is heated at reflux for one more hour. After cooling to room temperature methanol is added, and the mixture is heated to reflux for about 30 minutes to give the free base of the compound of formula I-B1.

A compound of formula I-B2 can be obtained as follows:

To a solution of a compound of formula XII in DMF is added sodium hydride at room temperature. After 30 minutes methyl iodide is added, and stirring is continued for about 2 h. Quenching with water is followed by extraction and purification to give a compound of formula XIII.

A mixture of a compound of formula XIII and of a THF solution of borane dimethylsulide complex in toluene is heated at reflux for about 20 h. After cooling to room temperature an aqueous hydrochloric acid solution is added, and the mixture is heated to reflux for about 30 minutes. The reaction mixture is cooled to room temperature, diluted with aqueous hydrochloric acid solution and washed with methyl tert.-butyl ether. The combined organic layers are extracted with aqueous hydrochloric acid solution. The combined aqueous layers are basified with aqueous sodium hydroxide solution and extracted with dichloromethane. The combined organic extracts are dried and concentrated in vacuo. It is obtained a compound of formula I-B2.

It has been shown that trans-derivatives, such as those described in WO2005/032464, do not have a dual activity or they have a very low dual activity, and therefore cannot share the advantages described above for the compounds of the present invention. In the table below are shown NK-1 and SERT activities of compounds of present cis-derivatives of formula I compared with structure-related trans-derivatives of formulas II, III and IV, encompassed by WO2005/032464:

| | |
|---|---|
| | I |
| 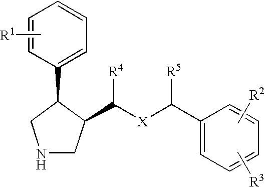 | |

| | |
|---|---|
| | II |
| 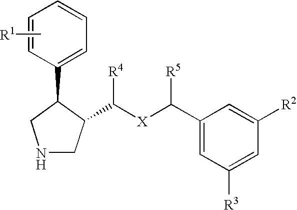 | |
| | III |
| 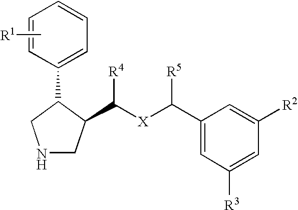 | |
| | IV |
| 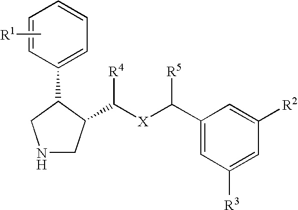 | |

| | | | | | | pKi | pKi | |
|---|---|---|---|---|---|---|---|---|
| R¹ | R² | R³ | R⁴ | R⁵ | X | hSERT | hNK1 | Example |
| 4-F | 3-CF₃ | 5-CF₃ | H | H | —O— | 8.56 | 6.53 | 1 of I |
| 3-F | 3-CF₃ | 5-CF₃ | H | H | —O— | 8.23 | 7.33 | 2 of I |
| 2-F | 3-CF₃ | 5-CF₃ | H | H | —O— | 8.46 | 7.30 | 3 of I |
| 4-Cl | 3-CF₃ | 5-CF₃ | H | H | —O— | 8.35 | 7.27 | 4 of I |
| 3-Cl | 3-CF₃ | 5-CF₃ | H | H | —O— | 8.09 | 7.50 | 5 of I |
| 2-Cl | 3-CF₃ | 5-CF₃ | H | H | —O— | 7.99 | 7.36 | 6 of I |
| 2-CH₃ | 3-CF₃ | 5-CF₃ | H | H | —O— | 8.12 | 7.54 | 7 of I |
| H | 3-CF₃ | 5-CF₃ | H | H | —O— | 8.31 | 7.36 | 8 of I |
| H | 3-Br | 5-Br | H | H | —O— | 8.23 | 7.38 | 9 of I |
| H | 3-CH₃ | 5-CH₃ | H | H | —O— | 8.34 | 6.59 | 10 of I |
| H | 3-CF₃ | 5-F | H | H | —O— | 8.24 | 6.65 | 11 of I |
| H | 3-CF₃ | H | H | H | —O— | 7.96 | 6.17 | 12 of I |
| H | 3-OCF₃ | H | H | H | —O— | 7.86 | 6.12 | 13 of I |
| H | 3-OCH₃ | 5-OCH₃ | H | H | —O— | 7.69 | 6.13 | 14 of I |
| H | 2-CF₃ | H | H | H | —O— | 7.15 | 6.19 | 15 of I |
| H | 3-CF₃ | 5-CF₃ | H | H | NH | 7.64 | 7.58 | 16 of I |
| H | 3-CF₃ | 5-CF₃ | H | H | N(CH₃) | 8.09 | 7.50 | 17 of I |
| 4-F | CF₃ | CF₃ | H | H | —O— | 6.93 | 8.12 | 1 of II |
| 4-F | CF₃ | CF₃ | H | 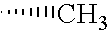 | —O— | 6.90 | 8.74 | 2 of II |
| 4-F | CF₃ | CF₃ | H | 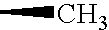 | —O— | 6.39 | 8.15 | 3 of II |
| 2-CH₃ | CF₃ | CF₃ | H | 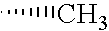 | —O— | 5.82 | 8.38 | 4 of II |
| 2-CH₃ | CF₃ | CF₃ | H | 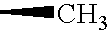 | —O— | 6.09 | 7.86 | 5 of II |
| 2-CH₃ | CF₃ | CF₃ | H | H | —O— | 6.28 | 7.90 | 6 of II |
| 3-Cl | CF₃ | CF₃ | H | H | —O— | 6.49 | 7.57 | 7 of II |
| 4-F | CF₃ | CF₃ | H | H | —O— | 6.35 | 7.90 | 1 of III |
| 4-F | CF₃ | CF₃ | H | 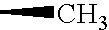 | —O— | 5.66 | 7.32 | 2 of III |
| 4-F | CF₃ | CF₃ | H | 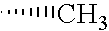 | —O— | 5.82 | 8.16 | 3 of III |
| 2-CH₃ | CF₃ | CF₃ | H | H | —O— | 6.64 | 8.43 | 4 of III |
| 2-CH₃ | CF₃ | CF₃ | H | 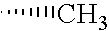 | —O— | 6.11 | 8.90 | 5 of III |
| 2-CH₃ | CF₃ | CF₃ | H | 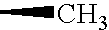 | —O— | 6.42 | 8.14 | 6 of III |
| 3-Cl | CF₃ | CF₃ | H | H | —O— | 6.13 | 7.05 | 7 of III |
| 4-Cl | CF₃ | CF₃ | H | H | —O— | 6.32 | 7.29 | 8 of III |
| 2-F | CF₃ | CF₃ | H | H | —O— | 6.96 | 8.10 | 9 of III |
| 4-F | CF₃ | CF₃ | H | H | —O— | 6.79 | 6.36 | 1 of IV |
| 3-F | CF₃ | CF₃ | H | H | —O— | 6.58 | 7.20 | 2 of IV |
| 2-CH₃ | CF₃ | CF₃ | H | 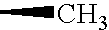 | —O— | 6.54 | 6.20 | 3 of IV |
| 2-CH₃ | CF₃ | CF₃ | H | 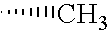 | —O— | 6.35 | 6.08 | 4 of IV |

Related compounds of formulas II and III have a high selectivity to the NK-1 receptor (not desired in the present case) and compounds of formula IV show an unexpectedly low level of activity on both receptors.

Furthermore, substitution on the N-atom in the pyrrolidine-ring, such as in the compounds of WO2005/032464, leads to a decrease of the SERT activity, as shown below:

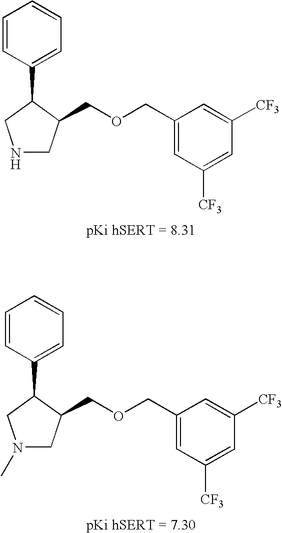

The data has been generated in accordance with the following assays:

### hSERT SPA Binding Assay

HEK-293 cells stably expressing recombinant human SERT are maintained with DMEM high glucose with 10% FBS, 300 μg/ml G418 and 2 mM L-Glutamine and incubated at 37° C. with 5% CO₂. Cells are released from culture flasks using PBS for 1-2 min. The cells are subsequently centrifuged at 1000 g's for 5 min and resuspended in PBS prior to being used in the membrane preparation.

Cells are homogenized using a Polytron in 50 mM Tris (pH 7.4). Centrifuged at 48,000×g for 15 min, and the pellet resuspended in fresh buffer. After a second centrifugation, the pellet is re-homogenized and resuspended in fresh buffer. Typically, membrane portions are aliquoted in 3 mg/ml (w:v). and stored at −80° C.

A serial dilution of test compounds in 50 mM Tris-HCl, 120 mM NaCl, KCl 5 mM (pH 7.4) is made in a white Optiplate (Packard) (100 μl/well) and the radioligand ³[H] Citalopram (Specific activity: 60-86 Ci/mmol, Final concentration: 1 nM) is added at 50 μl/well. Membrane and beads are prepared to a ratio of 5 μg:0.6 mg, with 0.6 mg PVT-WGA Amersham beads (Cat#RPQ0282V) added per well. 50 μl of the membrane/bead mixture is added to the assay plate for a final volume of 200 μl. The mixtures are allowed to stand at room temperature for one hour, and are then counted on a Packard TopCount.

The % inhibition is calculated for each compound tested (with 100% binding being the value obtained with the incubation of membrane/beads and radioligand in buffer without compound minus the non-specific binding measured in presence of 10 μM Fluoxetine). The concentration producing 50% inhibition (IC₅₀) is determined using an iterative non-linear curve fitting technique. The inhibition dissociation constant (Ki) of each compound is determined according to the method of Cheng-Prusoff, decribed in Biochem. Pharmacol., 1973, 22(23), pages 3099-3108.

### hNK-1 Binding Assay

The affinity of test compounds for the NK-1 receptor was evaluated at human NK-1 receptors in CHO cells transfected with the human NK-1 receptor using the Semliki virus expression system and radiolabelled with [³H]substance P (final concentration 0.6 nM). Binding assays were performed in HEPES buffer (50 mM, pH 7.4) containing BSA (0.04%) leupeptin (8 μg/ml), MnCl₂ (3 mM) and phosphoramidon (2 μM). Binding assays consisted of 250 μl of membrane suspension (1.25×10⁵ cells/assay tube), 125 μl of buffer of displacing agent and 125 μl of [³H]substance P. Displacement curves were determined with at least ten concentrations of the compound. The assay tubes were incubated for 60 min at room temperature after which time the tube contents were rapidly filtered under vacuum through GF/C filters presoaked for 60 min with PEI (0.3%) with 2×2 ml washes of HEPES buffer (50 mM, pH 7.4). The radioactivity retained on the filters was measured by scintillation counting. All assays were performed in duplicate in at least 2 separate experiments.

The inhibition dissociation constant (Ki) of each compound for NK1 is determined as described above for hSERT.

The following examples describe the preparation of compounds of formula I in more detail.

### Intermediate A

### (+)-3-(4-Fluoro-phenyl)-4-hydroxymethyl-pyrrolidine-1-carboxylic acid tert-butyl ester

### a) (Z)-3-(4-Fluoro-phenyl)-acrylic acid methyl ester

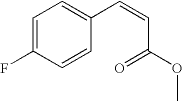

To a solution of 3.45 g (10.9 mmol) bis(2,2,2-trifluoroethyl) (methoxycarbonylmethyl)-phosphonate and 7.18 g (27.2 mmol) 18-crown-6 in 220 ml THF were added 11.9 ml (10.9 mmol) potassium hexamethyldisilazide solution (0.91 M in THF) at −78°. After 5 min 1.5 g (10.9 mmol) 4-fluorobenzaldehyde were added. The reaction mixture was stirred at −78° C. for 20 min and consequently allowed to warm to room temperature over a period of 1 h. Quenching with a saturated aqueous solution of ammonium chloride was followed by extraction with methyl tert.-butyl ether. The combined organic layers were washed with brine, dried over sodium sulfate and concentrated in vacuo. The crude product was purified by flash column chromatography (silica gel, heptane/ethyl acetate) to give 1.56 g (80%) of the title compound as a slightly yellow oil. MS m/e (%): 180 (M⁺, 68)

### b) (3RS,4RS)-1-Benzyl-4-(2-fluoro-phenyl)-pyrrolidine-3-carboxylic acid methyl ester

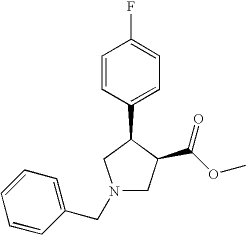

To a solution of 1.56 g (8.66 mmol) (Z)-3-(4-fluoro-phenyl)-acrylic acid methyl ester and 2.26 g (9.14 mmol) N-(methoxymethyl)-N-(trimethylsilylmethyl)benzylamine (96%, Aldrich) in 40 ml dichloromethane were added 0.066 ml (0.87 mmol) trifluoroacetic acid as a solution in 1 ml dichloromethane at 0° C. Another portion of 1.03 g (4.16 mmol) N-(methoxymethyl)-N-(trimethylsilylmethyl)benzylamine (96%, Aldrich) was added after one hour. After complete consumption of (Z)-3-(4-fluoro-phenyl)-acrylic acid methyl ester the reaction mixture was diluted with dichloromethane and washed with saturated sodium carbonate solution. The aqueous layer was extracted with two portions of dichloromethane. The combined organic layers were washed with brine, dried over sodium sulfate and concentrated in vacuo. The crude product was purified by flash column chromatography (silica gel, heptane/ethyl acetate) to give 2.36 g (87%) of the title compound as a slightly yellow oil. MS m/e (%): 314 (M+H⁺, 100)

### c) (+)-4-(4-Fluoro-phenyl)-pyrrolidine-1,3-dicarboxylic acid 1-tert-butyl ester 3-methyl ester

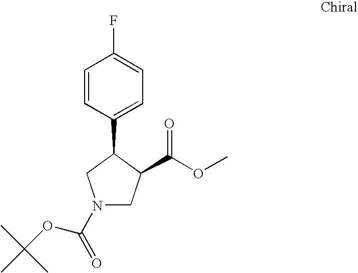

A solution of 7.65 g (24.4 mmol) (3RS,4RS)-1-benzyl-4-(2-fluoro-phenyl)-pyrrolidine-3-carboxylic acid methyl ester and 3.2 ml (29 mmol) 1-chloroethyl chloroformate in 80 ml 1,2-dichloroethane was heated at 50° C. over night. After cooling to room temperature the solvent was evaporated. The residue was redissolved in 80 ml MeOH and the resulting solution was heated at reflux for 30 min. The reaction mixture was concentrated in vacuo. The residual hydrochloride salt was redissolved in 50 ml THF. The mixture was cooled to 0° C. and treated with 3.4 ml (24 mmol) triethylamine and a solution of 6.4 g (29 mmol) di-tert.-butyl dicarbonate in 30 ml THF. After stirring for 90 min the reaction mixture was diluted with methyl tert.-butyl ether and water. The pH of the aqueous layer was adjusted to 2 by the addition of 2 M aqueous hydrochloric acid solution. The layers were separated and the aqueous layer was extracted with two more portions of methyl tert.-butyl ether. The combined organic layers were dried over sodium sulfate, concentrated in vacuo and purified by flash chromatography to give 5.82 g (74%) of (3RS,4RS)-4-(4-fluoro-phenyl)-pyrrolidine-1,3-dicarboxylic acid 1-tert-butyl ester 3-methyl ester as a slightly yellow oil.

The enantiomers were separated by chiral HPLC (Chiralpak AD; heptane/EtOH 95:5) to give 2.36 g of the title compound as slightly yellow oil.

MS m/e (%): 324 (M+H⁺, 11), 346 (M+Na⁺, 29)

[α]_{D}=+54.98 (c=0.422, CHCl₃)

### d) (+)-4-(4-Fluoro-phenyl)-pyrrolidine-1,3-dicarboxylic acid 1-tert-butyl ester

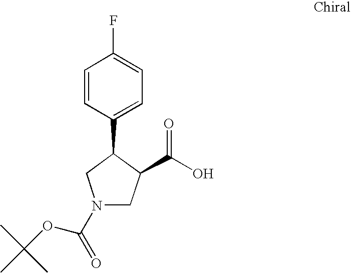

A mixture of 1.79 g (5.54 mmol) (+)-4-(4-fluoro-phenyl)-pyrrolidine-1,3-dicarboxylic acid 1-tert-butyl ester 3-methyl ester and 15 ml of a 2 M aqueous hydrochloric acid solution was heated at reflux for 4 h. The reaction mixture was cooled to 0° C. and basified by the addition of 3.6 ml of a 32% aqueous solution of sodium hydroxide. Dilution with 10 ml 1,4-dioxane was followed by addition of a solution of 2.42 g (11.1 mmol) di-tert.-butyl dicarbonate in 5 ml 1,4-dioxane. After completed addition the reaction mixture was allowed to slowly warm to room temperature over night. The mixture was extracted methyl tert.-butyl ether at pH 8. The organic layer was extracted with a 1 M aqueous sodium hydroxide solution. The combined aqueous layers were acidified to pH 1 by the addition of and ice cold 2 M aqueous hydrochloric acid solution at 0° C. and extracted with three portions of ethyl acetate. The combined organic layers were dried over sodium sulfate, concentrated in vacuo and purified by flash chromatography to give 1.55 g (91%) of the title compound as an off-white solid.

MS m/e (%): 308 ([M−H⁺]⁻, 100)

[α]_{D}=+31.32 (c=0.3768, CHCl₃)

### e) (+)-3-(4-Fluoro-phenyl)-4-hydroxymethyl-pyrrolidine-1-carboxylic acid tert-butyl ester

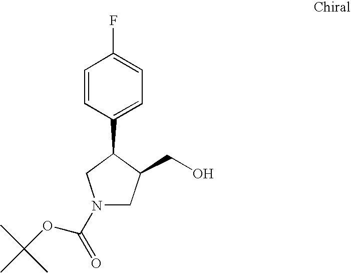

To a solution of 0.75 g (2.4 mmol) (+)-4-(4-fluoro-phenyl)-pyrrolidine-1,3-dicarboxylic acid 1-tert-butyl ester in 12 ml THF were added 2.42 ml (4.84 mmol) of a 2 M borane dimethylsulfide complex solution in THF at 0° C. The mixture was stirred for 15 min at 0° C. and then for 5 h at room temperature. After cooling to 0° C. the reaction was quenched by the addition of methanol. Stirring was continued until no evolution of gas was observed any more. The reaction mixture was concentrated in vacuo. The residue was purified by flash chromatography to give 0.61 g (85%) of the title compound as a colorless amorphous solid.

MS m/e (%): 296 (M+H⁺, 24)

[α]_{D}=+62.40 (c=0.423, CHCl₃)

### Intermediate B

### (+)-3-(3-Fluoro-phenyl)-4-hydroxymethyl-pyrrolidine-1-carboxylic acid tert-butyl ester

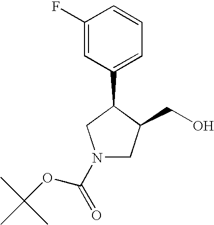

The title compound was obtained as a light yellow viscous oil in comparable yields after flash chromatography according to the procedures described above for the preparation of (+)-3-(4-fluoro-phenyl)-4-hydroxymethyl-pyrrolidine-1-carboxylic acid tert-butyl ester using 3-fluorobenzaldehyde instead of 4-fluorobenzaldehyde in step a).

MS m/e (%): 296 (M+H⁺, 7)

[α]_{D}=+61.86 (c=0.892, CHCl₃)

### Intermediate C

### (+)-3-(2-Fluoro-phenyl)-4-hydroxymethyl-pyrrolidine-1-carboxylic acid tert-butyl ester

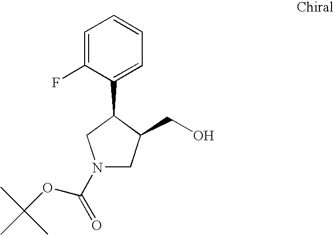

The title compound was obtained as an off-white amorphous solid in comparable yields after flash chromatography according to the procedures described above for the preparation of (+)-3-(4-fluoro-phenyl)-4-hydroxymethyl-pyrrolidine-1-carboxylic acid tert-butyl ester using 2-fluorobenzaldehyde instead of 4-fluorobenzaldehyde in step a). In step c) enantiomerically pure (+)-1-benzyl-4-(2-fluoro-phenyl)-pyrrolidine-3-carboxylic acid methyl ester was used after separation of the racemic mixture obtained in step b) by preparative HPLC (Chiralcel OD, heptane/EtOH 99:1).

MS m/e (%): 295 (M⁺, 7)

[α]_{D}=+72.54(c=0.531, CHCl₃)

### Intermediate D

### (+)-3-(4-Chloro-phenyl)-4-hydroxymethyl-pyrrolidine-1-carboxylic acid tert-butyl ester

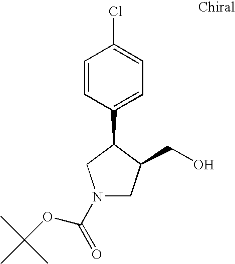

The title compound was obtained as a colorless amorphous solid in comparable yields after flash chromatography according to the procedures described above for the preparation of (+)-3-(4-fluoro-phenyl)-4-hydroxymethyl-pyrrolidine-1-carboxylic acid tert-butyl ester using 4-chlorobenzaldehyde instead of 4-fluorobenzaldehyde in step a).

MS m/e (%): 312 (M+H⁺, 75)

[α]_{D}=+63.80 (c=0.635, CHCl₃)

### Intermediate E

### (+)-3-(3-Chloro-phenyl)-4-hydroxymethyl-pyrrolidine-1-carboxylic acid tert-butyl ester

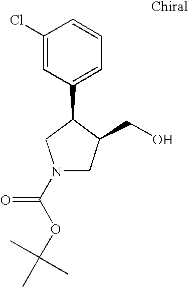

The title compound was obtained as a colorless amorphous solid in comparable yields after flash chromatography according to the procedures described above for the preparation of (+)-3-(4-fluoro-phenyl)-4-hydroxymethyl-pyrrolidine-1-carboxylic acid tert-butyl ester using 3-chlorobenzaldehyde instead of 4-fluorobenzaldehyde in step a). In step c) enantiomerically pure (+)-1-benzyl-4-(3-chloro-phenyl)-pyrrolidine-3-carboxylic acid methyl ester was used after separation of the racemic mixture obtained in step b) by preparative HPLC (Chiralcel OD, heptane/EtOH 98:2).

MS m/e (%): 312 (M+H⁺, 18)

[α]_{D}=+69.19(c=0.597, CHCl₃)

### Intermediate F

### (+)-3-(2-Chloro-phenyl)-4-hydroxymethyl-pyrrolidine-1-carboxylic acid tert-butyl ester

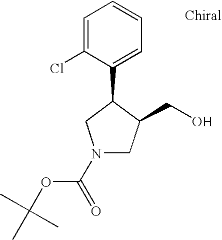

The title compound was obtained as a light yellow amorphous solid in comparable yields after flash chromatography according to the procedures described above for the preparation of (+)-3-(4-fluoro-phenyl)-4-hydroxymethyl-pyrrolidine-1-carboxylic acid tert-butyl ester using 2-chlorobenzaldehyde instead of 4-fluorobenzaldehyde in step a).

MS m/e (%): 312 (M+H⁺, 100)

[α]_{D}=+90.85 (c=0.790, CHCl₃)

### Intermediate G

### (+)-3-Hydroxymethyl-4-o-tolyl-pyrrolidine-1-carboxylic acid tert-butyl ester

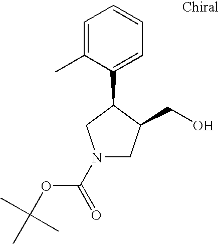

The title compound was obtained as a light yellow amorphous solid in comparable yields after flash chromatography according to the procedures described above for the preparation of (+)-3-(4-fluoro-phenyl)-4-hydroxymethyl-pyrrolidine-1-carboxylic acid tert-butyl ester using o-tolylbenzaldehyde instead of 4-fluorobenzaldehyde in step a).

MS m/e (%): 292 (M+H⁺, 100)

[α]_{D}=+77.97 (c=1.049, CHCl₃)

### Intermediate H

### (+)-3-Hydroxymethyl-4-phenyl-pyrrolidine-1-carboxylic acid tert-butyl ester

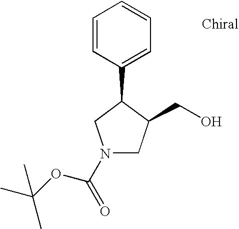

The title compound is obtained as a colorless amorphous solid in comparable yields after flash chromatography according to the procedures described above for the preparation of (+)-3-(4-fluoro-phenyl)-4-hydroxymethyl-pyrrolidine-1-carboxylic acid tert-butyl ester using benzaldehyde instead of 4-fluorobenzaldehyde in step a).

MS m/e (%): 278 (M+H⁺, 10)

[α]_{D}=+71.84 (c=0.877, CHCl₃)

### EXAMPLE 1

### (+)-3-(3,5-Bis-trifluoromethyl-benzyloxymethyl)-4-(4-fluoro-phenyl)-pyrrolidine hydrochloride

### a) (+)-3-(3,5-Bis-trifluoromethyl-benzyloxymethyl)-4-(4-fluoro-phenyl)-pyrrolidine-1-carboxylic acid tert-butyl ester

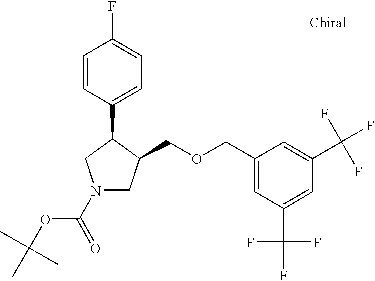

To a solution of 0.17 g (0.58 mmol) (+)-3-(4-fluoro-phenyl)-4-hydroxymethyl-pyrrolidine-1-carboxylic acid tert-butyl ester (Intermediate A) in 6 ml DMF were added 0.029 g (0.60 mmol) sodium hydride (50% in mineral oil) at 0° C. The reaction mixture was allowed to warm to room temperature. After 1 h 0.11 ml (0.60 mmol) 3,5-bis(trifluoromethyl)benzyl bromide were added. Stirring at room temperature for 2.5 h was followed by quenching with 10 ml of water and extraction with three portions of methyl tert.-butylether. The combined organic layers were washed with brine, dried over sodium sulfate and concentrated in vacuo. The crude product was purified by flash column chromatography (silica gel, heptane/ethyl acetate) to give 0.183 g (61%) of the title compound as an amorphous solid.

MS m/e (%): 522 (M+H⁺, 100)

[α]_{D}=+20.20 (c=1.129, CHCl₃)

### b) (+)-3-(3,5-Bis-trifluoromethyl-benzyloxymethyl)-4-(4-fluoro-phenyl)-pyrrolidine hydrochloride

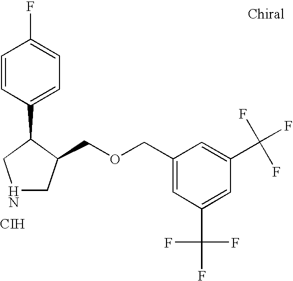

A solution of 0.17 g (0.33 mmol) (+)-3-(3,5-bis-trifluoromethyl-benzyloxymethyl)-4-(4-fluoro-phenyl)-pyrrolidine-1-carboxylic acid tert-butyl ester in 2.65 ml of a 1.25 M solution of hydrochloric acid (3.3 mmol) in methanol was stirred for 30′ at 50° C. The reaction mixture was concentrated to dryness to give 0.14 g (92%) of the title compound as a colorless solid.

MS m/e (%): 422 (M+H⁺, 100)

[α]_{D}=+29.36 (c=0.504, CHCl₃)

### EXAMPLE 2

### (+)-3-(3,5-Bis-trifluoromethyl-benzyloxymethyl)-4-(3-fluoro-phenyl)-pyrrolidine hydrochloride

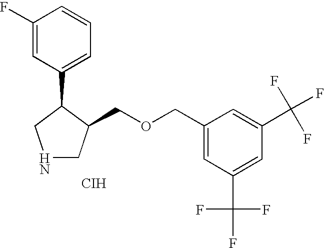

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate B instead of Intermediate A in step a).

MS m/e (%): 422 (M+H⁺, 100)

[α]_{D}=+31.60 (c=0.446, CHCl₃)

### EXAMPLE 3

### (+)-3-(3,5-Bis-trifluoromethyl-benzyloxymethyl)-4-(2-fluoro-phenyl)-pyrrolidine hydrochloride

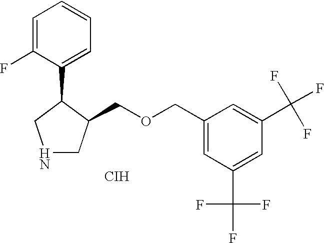

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate C instead of Intermediate A in step a).

MS m/e (%): 422 (M+H⁺, 100)

[α]_{D}=+40.56 (c=0.385, CHCl₃)

### EXAMPLE 4

### (+)-3-(3,5-Bis-trifluoromethyl-benzyloxymethyl)-4-(4-chloro-phenyl)-pyrrolidine hydrochloride

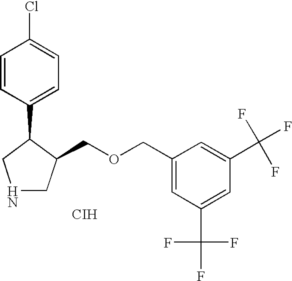

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate D instead of Intermediate A in step a).

MS m/e (%): 438 (M+H⁺, 100)

[α]_{D}=+29.97 (c=0.394, CHCl₃)

### EXAMPLE 5

### (+)-3-(3,5-Bis-trifluoromethyl-benzyloxymethyl)-4-(3-chloro-phenyl)-pyrrolidine hydrochloride

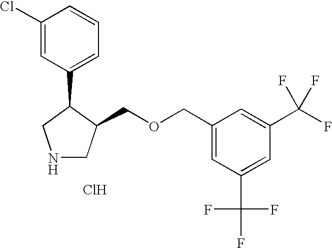

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate E instead of Intermediate A in step a).

MS m/e (%): 438 (M+H⁺, 100)

[α]_{D}=+35.06 (c=0.685, CHCl₃)

### EXAMPLE 6

### (+)-3-(3,5-Bis-trifluoromethyl-benzyloxymethyl)-4-(2-chloro-phenyl)-pyrrolidine hydrochloride

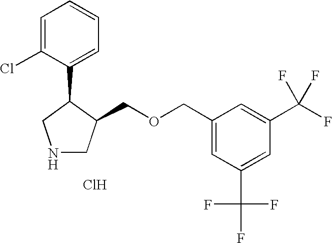

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate F instead of Intermediate A in step a).

MS m/e (%): 438 (M+H⁺, 100)

[α]_{D}=+53.72 (c=0.672, CHCl₃)

### EXAMPLE 7

### (+)-3-(3,5-Bis-trifluoromethyl-benzyloxymethyl)-4-o-tolyl-pyrrolidine hydrochloride

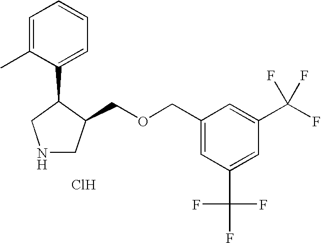

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate G instead of Intermediate A in step a).

MS m/e (%): 418 (M+H⁺, 100)

[α]_{D}=+47.53 (c=0.962, CHCl₃)

### EXAMPLE 8

### (+)-3-(3,5-Bis-trifluoromethyl-benzyloxymethyl)-4-phenyl-pyrrolidine hydrochloride

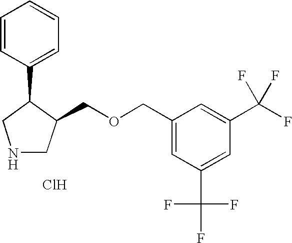

The title compound was obtained as a light yellow solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A in step a).

MS m/e (%): 404 (M+H⁺, 100)

[α]_{D}=+22.79 (c=0.592, CHCl₃)

### EXAMPLE 9

### (+)-3-(3,5-Dibromo-benzyloxymethyl)-4-phenyl-pyrrolidine hydrochloride

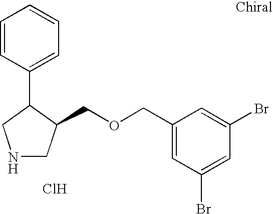

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A in step a).

MS m/e (%): 426 (M+H⁺, 100)

[α]_{D}=+39.42 (c=0.416, CHCl₃)

### EXAMPLE 10

### (+)-3-(3,5-Dimethyl-benzyloxymethyl)-4-phenyl-pyrrolidine hydrochloride

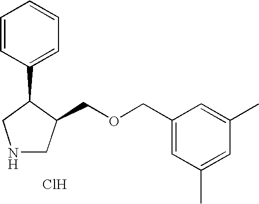

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A and 3,5-dimethylbenzyl bromide instead of 3,5-bis(trifluoromethyl)benzyl bromide in step a).

MS m/e (%): 296 (M+H⁺, 100)

[α]_{D}=+58.67 (c=0.375, CHCl₃)

### EXAMPLE 11

### (+)-3-(3-Fluoro-5-trifluoromethyl-benzyloxymethyl)-4-phenyl-pyrrolidine hydrochloride

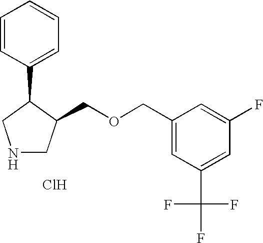

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A and 3-fluoro-5-(trifluoromethyl)benzyl bromide instead of 3)-5-bis(trifluoromethyl)benzyl bromide in step a). MS m/e (%): 354 (M+H⁺, 100)

[α]_{D}=+41.40 (c=0.722, CHCl₃)

### EXAMPLE 12

### (+)-3-Phenyl-4-(3-trifluoromethyl-benzyloxymethyl)-pyrrolidine hydrochloride

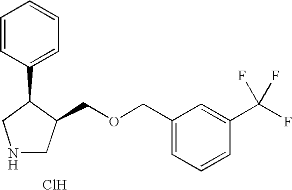

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A and 3-(trifluoromethyl)benzyl bromide instead of 3,5-bis(trifluoromethyl)benzyl bromide in step a).

MS m/e (%): 336 (M+H⁺, 100)

[α]_{D}=+41.84 (c=0.619, CHCl₃)

### EXAMPLE 13

### (+)-3-Phenyl-4-(3-trifluoromethoxy-benzyloxymethyl)-pyrrolidine hydrochloride

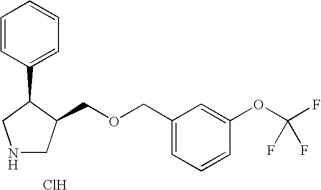

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A and 3-(trifluoromethoxy)benzyl bromide instead of 3,5-bis(trifluoromethyl)benzyl bromide in step a).

MS m/e (%): 352 (M+H⁺, 100)

[α]_{D}=+42.31 (c=0.643, CHCl₃)

### EXAMPLE 14

### (+)-3-(3,5-Dimethoxy-benzyloxymethyl)-4-phenyl-pyrrolidine hydrochloride

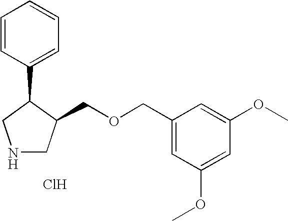

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A and 3,5-(dimethoxy)benzyl bromide instead of 3,5-bis(trifluoromethyl)benzyl bromide in step a).

MS m/e (%): 328 (M+H⁺, 100)

[α]_{D}=+43.53 (c=0.611, CHCl₃)

### EXAMPLE 15

### (+)-3-Phenyl-4-(2-trifluoromethyl-benzyloxymethyl)-pyrrolidine hydrochloride

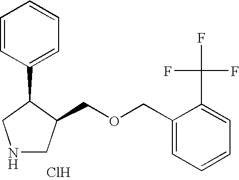

The title compound was obtained as an off-white solid in comparable yields according to the procedures described above for the preparation of (+)-3-(3,5-bis-trifluoromethyl-benzyloxymethyl)-4-(4-fluoro-phenyl)-pyrrolidine hydrochloride using Intermediate H instead of Intermediate A and 2-(trifluoromethyl)benzyl bromide instead of 3,5-bis(trifluoromethyl)benzyl bromide in step a).

MS m/e (%): 336 (M+H⁺, 100)

[α]_{D}=+40.40 (c=0.654, CHCl₃)

### EXAMPLE 16

### (+)-(3,5-Bis-trifluoromethyl-benzyl)-((3R,4R)-4-phenyl-pyrrolidin-3-ylmethyl)-amine dihydrochloride

### a) (+)-(3R,4R)-3-Aminomethyl-4-phenyl-pyrrolidine-1-carboxylic acid tert-butyl ester

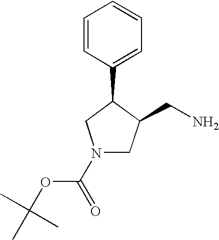

To a solution of 2.00 g (7.21 mmol) (+)-(3R,4R)-3-hydroxymethyl-4-phenyl-pyrrolidine-1-carboxylic acid tert-butyl ester (Intermediate H) and 1.11 ml (7.94 mmol) triethylamine in 35 ml dichloromethane were added dropwise at 0° C. 0.59 ml (7.6 mmol) methanesulfonyl chloride. After completed addition the reaction mixture was allowed to warm to room temperature during 30 minutes. Quenching with water was followed by extraction with two portions of methyl tert.-butyl ether. The combined organic layers were washed with water and brine, dried over sodium sulfate and concentrated in vacuo to give 2.57 g (100%) of crude (3R,4R)-3-methanesulfonyloxymethyl-4-phenyl-pyrrolidine-1-carboxylic acid tert-butyl ester, which was used in the next step without purification.

A mixture of 1.30 g (3.66 mmol) crude (3R,4R)-3-methanesulfonyloxymethyl-4-phenylpyrrolidine-1-carboxylic acid tert-butyl ester and 357 mg (5.49 mmol) sodium azide in 8 ml dimethylsulfoxide was heated at 120° C. for 5 h. The mixture was cooled to room temperature, diluted with a 0.2 M aqueous solution of sodium carbonate and extracted with two portions of methyl tert.-butyl ether. The combined organic layers were washed with water and brine, dried over sodium sulfate and concentrated in vacuo to give 1.07 g (97%) of crude (3R,4R)-3-azidomethyl-4-phenyl-pyrrolidine-1-carboxylic acid tert-butyl ester This material was dissolved in 17.5 ml THF and treated with 975 mg (3.71 mmol) triphenylphosphine and 1.2 ml of water. After stirring at room temperature for 20 h the reaction mixture was diluted with methyl tert.-butyl ether and washed with three portions of a half-saturated aqueous solution of ammonium chloride. The combined aqueous layers were basified by the addition of 2 M aqueous sodium hydroxide solution and extracted with three portions of methyl tert.-butyl ether. The combined organic layers were dried over sodium sulfate and concentrated in vacuo to give 0.74 g (75%) of the crude title compound as a colorless amorphous solid.

MS m/e (%):277 (M+H⁺, 21)

[α]_{D}=+52.30 (c=0.579, CHCl₃)

### b) (3S,4R)-3-[(3,5-Bis-trifluoromethyl-benzoylamino)-methyl]-4-phenyl-pyrrolidine-1-carboxylic acid tert-butyl ester

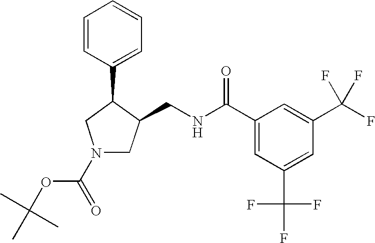

To a solution of 0.36 g (1.3 mmol) (+)-(3R,4R)-3-aminomethyl-4-phenyl-pyrrolidine-1-carboxylic acid tert-butyl ester and 0.22 ml (1.6 mmol) triethylamine in 6.5 ml dichloromethane were added 0.26 ml (1.4 mmol) 3,5-bis(trifluoromethyl)benzoyl chloride at 0° C. After completed addition the reaction mixture was allowed to warm to room temperature and stirred for one hour. Quenching with water was followed by basification with 1 M aqueous sodium hydroxide solution and extraction with three portions of methyl tert.-butyl ether. The combined organic layers were washed with brine, dried over sodium sulfate and concentrated in vacuo. Flash column chromatography gave 0.62 g (92%) of the title compound as a white solid.

MS m/e (%): 517 (M+H⁺, 21)

### c)-(+)-(3,5-Bis-trifluoromethyl-benzyl)-((3R,4R)-4-phenyl-pyrrolidin-3-ylmethyl)-amine dihydrochloride

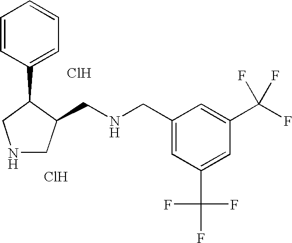

A mixture of 0.29 g (0.57 mmol) (3S,4R)-3-[(3,5-bis-trifluoromethyl-benzoylamino)-methyl]-4-phenyl-pyrrolidine-1-carboxylic acid tert-butyl ester and 0.28 ml of a 2 M THF solution of borane dimethylsulide complex (0.56 mmol) in 6 ml toluene was heated at reflux for 20 h. After addition of another portion of 0.28 ml of a 2 M THF solution of borane dimethylsulfide complex (0.56 mmol) the mixture was heated at reflux for one more hour. After cooling to room temperature 1 ml of methanol was added, and the mixture was heated to reflux for 30 minutes. Concentration in vacuo was followed by flash column chromatography to give the free base of the title compound. Dissolution in a 1.25 M solution of hydrochloric acid and concentration in vacuo gave 91 mg (34%) of the title compound as a light yellow solid.

MS m/e (%): 403 (M+H⁺, 100)

[α]_{D}=+12.60 (c=0.500, MeOH)

### EXAMPLE 17

(3,5-Bis-trifluoromethyl-benzyl)-methyl-((3R,4R)-4-phenyl-pyrrolidin-3-ylmethyl)-amine dihydrochloride

### a) (3S,4R)-3-[(3,5-Bis-trifluoromethyl-benzoyl)-methyl-amino]-methyl]-4-phenyl-pyrrolidine-1-carboxylic acid tert-butyl ester

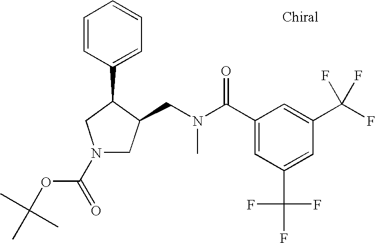

To a solution of 0.20 g (0.39 mmol) (3S,4R)-3-[(3,5-bis-trifluoromethyl-benzoylamino)-methyl]-4-phenyl-pyrrolidine-1-carboxylic acid tert-butyl ester (Example 20 of I b)) in 4 ml DMF were added 20 mg (0.42 mmol) sodium hydride (50% in mineral oil) at room temperature. After 30 minutes 0.027 ml (0.42 mmol) methyl iodide were added, and stirring was continued for 2 h. Quenching with water was followed by extraction with two portions of methyl tert.-butyl ether. The combined organic layers were washed with water and brine, dried over sodium sulfate and concentrated in vacuo. Flash column chromatography gave 0.19 g (93%) of the title compound as a white solid.

MS m/e (%):531 (M+H⁺, 22)

### b) (3,5-Bis-trifluoromethyl-benzyl)-methyl-((3R,4R)-4-phenyl-pyrrolidin-3-ylmethyl)-amine dihydrochloride

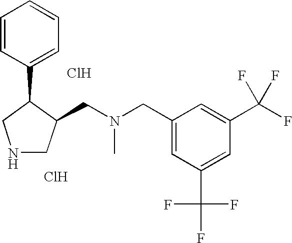

A mixture of 0.12 g (0.23 mmol) (3S,4R)-3-{[(3,5-bis-trifluoromethyl-benzoyl)-methyl-amino]-methyl}-4-phenyl-pyrrolidine-1-carboxylic acid tert-butyl ester and 0.11 ml of a 2 M THF solution of borane dimethylsulide complex (0.22 mmol) in 2 ml toluene was heated at reflux for 20 h. After cooling to room temperature a 2 M aqueous hydrochloric acid solution was added, and the mixture was heated to reflux for 30 minutes. The reaction mixture was cooled to room temperature, diluted with 1 M aqueous hydrochloric acid solution and washed with two portions of methyl tert-.butyl ether. The combined organic layers were extracted with 1 M aqueous hydrochloric acid solution. The combined aqueous layers were basified with 2 M aqueous sodium hydroxide solution and extracted with three portions of dichloromethane. The combined organic extracts were dried over sodium sulfate and concentrated in vacuo. Flash column chromatography gave the free base of the title compound. This material was purified further by trituration with warm methyl tert-.butyl ether followed by filtration and concentration of the filtrate in vacuo. Dissolution in a 1.25 M solution of hydrochloric acid and concentration in vacuo gave 55 mg (50%) of the title compound as a white solid.

MS m/e (%):417 (M+H⁺, 100)

1. A cis-derivative of formula I 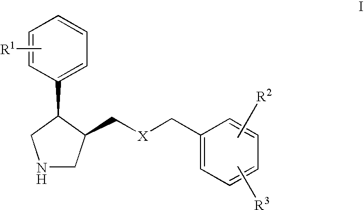
wherein
R¹ is hydrogen, halogen or lower alkyl;
R² and R³ are each independently hydrogen, halogen, CN, lower alkyl substituted by halogen or lower alkoxy substituted by halogen;
X is —O— or —N(R)—; and
R is hydrogen or lower alkyl;
or a pharmaceutically acceptable acid addition salt thereof.

2. A compound of claim 1 having formula IA 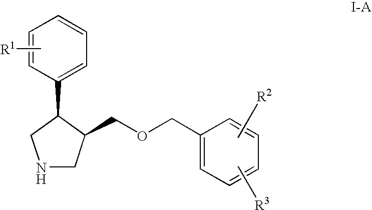
wherein
R¹ is hydrogen, halogen or lower alkyl; and
R² and R³ are each independently hydrogen, halogen, CN, lower alkyl substituted by halogen or lower alkoxy substituted by halogen;
or a pharmaceutically acceptable acid addition salt thereof.

3. A compound of claim 2, selected from the group consisting of
(+)-3-(3,5-bis-trifluoromethyl-benzyloxymethyl)-4-(4-fluoro-phenyl)-pyrrolidine,
(+)-3-(3,5-bis-trifluoromethyl-benzyloxymethyl)-4-(3-fluoro-phenyl)-pyrrolidine,
(+)-3-(3,5-bis-trifluoromethyl-benzyloxymethyl)-4-(2-fluoro-phenyl)-pyrrolidine,
(+)-3-(3,5-bis-trifluoromethyl-benzyloxymethyl)-4-(4-chloro-phenyl)-pyrrolidine,
(+)-3-(3,5-bis-trifluoromethyl-benzyloxymethyl)-4-(3-chloro-phenyl)-pyrrolidine,
(+)-3-(3,5-bis-trifluoromethyl-benzyloxymethyl)-4-(2-chloro-phenyl)-pyrrolidine,
(+)-3-(3,5-bis-trifluoromethyl-benzyloxymethyl)-4-o-tolyl-pyrrolidine,
(+)-3-(3,5-bis-trifluoromethyl-benzyloxymethyl)-4-phenyl-pyrrolidine, and
3-(3,5-dibromo-benzyloxymethyl)-4-phenyl-pyrrolidine.

4. A compound of claim 2, selected from the group consisting of
(+)-3-(3,5-dimethyl-benzyloxymethyl)-4-phenyl-pyrrolidine,
(+)-3-(3-fluoro-5-trifluoromethyl-benzyloxymethyl)-4-phenyl-pyrrolidine,
(+)-3-phenyl-4-(3-trifluoromethyl-benzyloxymethyl)-pyrrolidine,
(+)-3-(3,5-difluoro-benzyloxymethyl)-4-phenyl-pyrrolidine,
(+)-3-phenyl-4-(3-trifluoromethoxy-benzyloxymethyl)-pyrrolidine,
(+)-3-(3-difluoromethoxy-benzyloxymethyl)-4-phenyl-pyrrolidine,
(+)-3-(3,5-dimethoxy-benzyloxymethyl)-4-phenyl-pyrrolidine,
(+)-3-(4-phenyl-pyrrolidin-3-ylmethoxymethyl)-benzonitrile, phenyl-4-(4-trifluoromethyl-benzyloxymethyl)-pyrrolidine and
(+)-3-phenyl-4-(2-trifluoromethyl-benzyloxymethyl)-pyrrolidine.

5. A compound of claim 1 having formula IB 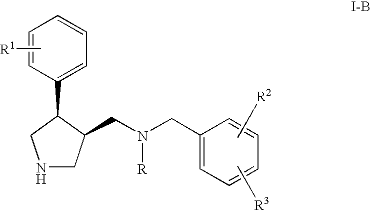
according to claim 1,
wherein
R¹ is hydrogen, halogen or lower alkyl;
R² and R³ are each independently hydrogen, halogen, CN, lower alkyl substituted by halogen or lower alkoxy substituted by halogen; and
R is hydrogen or lower alkyl;
or a pharmaceutically acceptable acid addition salt thereof.

6. A compound of claim 5, selected from the group consisting of
(+)-(3,5-bis-trifluoromethyl-benzyl)-((3R,4R)-4-phenyl-pyrrolidin-3-ylmethyl)-amine dihydrochloride and
(3,5-bis-trifluoromethyl-benzyl)-methyl-((3R,4R)-4-phenyl-pyrrolidin-3-ylmethyl)-amine dihydrochloride.

7. A compound of claim 5, wherein R is hydrogen.

8. The compound of claim 5, wherein R is lower alkyl.

9. A compound of claim 1, wherein R¹ is hydrogen.

10. A compound of claim 1, wherein R¹ is halogen.

11. A compound of claim 1, wherein R¹ is lower alkyl.

12. A compound of claim 11, wherein R¹ is methyl.

13. A compound of claim 1, wherein at least one of R² and R³ is hydrogen.

14. A compound of claim 1, wherein at least one of R² and R³ is lower alkyl.

15. A compound of claim 14, wherein at least one of R² and R³ is methyl.

16. A compound of claim 1, wherein at least one of R² and R³ is lower alkyl substituted by halogen.

17. A compound of claim 16, wherein at least one of R² and R³ is CF₃.

18. A compound of claim 1, wherein at least one of R² and R³ is halogen.

19. A compound of claim 1, wherein at least one of R² and R³ is lower alkoxy.

20. A compound of claim 19, wherein at least one of R² and R³ is OCH₃.

21. A compound of claim 19, wherein at least one of R² and R³ is lower alkoxy substituted by halogen.

22. A compound of claim 21, wherein at least one of R² and R³ is OCF₃.

23. A pharmaceutical composition comprising a therapeutically effective amount of a compound of formula I 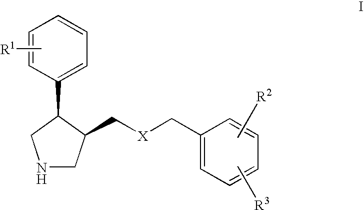
wherein
R¹ is hydrogen, halogen or lower alkyl;
R² and R³ are each independently hydrogen, halogen, CN, lower alkyl substituted by halogen or lower alkoxy substituted by halogen;
X is —O— or —N(R)—; and
R is hydrogen or lower alkyl;
or a pharmaceutically acceptable acid addition salt thereof.
